(19) United States
(12) Patent Application Publication
(10) Pub. No.: US 2024/0312368 A1
(43) Pub. Date: Sep. 19, 2024

(54) METHOD FOR MANUFACTURING ANATOMICAL MODELS ADAPTED TO SIMULATE ORGANS OR PARTS OF ORGANS OF A PATIENT

(71) Applicant: Universita' Degli Studi Di Milano, Milano (IT)

(72) Inventors: Paolo MILANI, Milano (IT); Maurizio VERTEMATI, Milano (IT); Francesco CAVALIERE, Milano (IT); Tommaso SANTANIELLO, Milano (IT); Lorenzo GENTILI, Milano (IT); Somone CASSIN, Milano (IT); Francesco RIZZETTO, Milano (IT)

(21) Appl. No.: 18/571,798

(22) PCT Filed: Jun. 10, 2022
(86) PCT No.: PCT/IB2022/055413

§ 371 Date: Dec. 19, 2023

(30) Foreign Application Priority Data
Jun. 22, 2021 (IT) 102021000016277

(51) Int. Cl. G09B 23/30; B33Y 10/00; B33Y 50/00

(52) U.S. Cl. CPC G09B 23/30; B33Y 10/00; B33Y 50/00

(57) ABSTRACT

The invention relates to a method for manufacturing anatomical models adapted to simulate organs or parts of organs of a patient. The method includes selecting elements of an anatomical structure of an organ or part of an organ from processed images to obtain a three-dimensional computerized model representing the three-dimensional computerized model by a file having a valid format usable in a three-dimensional printing process; selecting at least one material from a first software library of usable material and at least one manufacturing method from a second software library of manufacturing methods based on a parameter representative of a hardness measurement of said organ; modifying the file representative of the three-dimensional computerized model based on the at least one material and the at least one manufacturing method; using the modified file to perform the process for manufacturing the anatomical model by a three-dimensional printing unit.

### FIELD OF APPLICATION

The present invention relates in general to the field of medicine. The invention also concentrates on the field of the industry dedicated to making anatomical models for use in teaching or other medical disciplines, such as planning and simulating surgical procedures.

In particular, the invention relates to an innovative method for manufacturing anatomical models adapted to simulate organs or parts of organs of a patient.

### PRIOR ART

As known, a preoperative planning phase of a surgical procedure assumes an important role in modern surgery because it enables surgeons to optimize the surgical results and prevent complications during surgical procedures. Correct preoperative planning also allows for shorter surgery times, decreasing postoperative stress and even intraoperative blood loss.

Standard preoperative planning is performed by analyzing physical radiographic images or by using two-dimensional (2D) digital systems, which allow said radiographic images to be viewed and enlarged at an electronic terminal, and to be shared. More recently, it is known to employ three-dimensional (3D) reconstruction from computed tomography (CT) which has proven useful in surgical planning of complex bone fractures. Furthermore, several preoperative planning software solutions are known. Most of said solutions are used by surgeons, before surgery, from stations located at a distance from the operating room.

Currently, three-dimensional printing or 3D printing is often used for preoperative planning purposes. As known, 3D printing is a form of rapid prototyping/additive manufacturing useful for the rapid manufacturing of customized models. Indeed, customized models can be generated using Computer-Aided Design (CAD) programs, a 3D scanner, and/or photogrammetry software. Customized models can be manufactured using a 3D printer.

In the medical-surgical field, the terms “3D printing” or “rapid prototyping” relates to a set of technologies for producing physical parts of the human body starting from digital descriptions of said parts. Some uses of such technologies include the production of anatomical parts, such as bones, for research and clinical applications, or the development of medical products. Digital descriptions of the physical parts to be reproduced include output data from appropriate software configured to generate a 3D digital model. An example of such software is CAD software.

The starting point in the reproduction of physical models of human body parts usable for pre-surgical training and/or education is the development of a 3D virtual model, which represents the specific target anatomy of the patient.

Said 3D virtual model is obtained starting from medical images (CT-Computerized Tomography/NMR-Nuclear Magnetic Resonance Imaging/radiography) acquired in semiautomatic or manual mode.

Said 3D virtual model is, successively, processed by a first software capable of parameterizing the model itself according to a given scale, physical, dimensional and morphological factors, enabling its physical making at the same time. In more detail, a model or CAD file of the object to be manufactured is used to proceed with additive manufacturing by means of 3D printing.

Said CAD file is, successively, converted to a .stl (STereo Lithography interface format) file in which the surface of the object to be reproduced is represented/discretized through a network of triangles or polygons.

Afterwards, the .stl format file is converted into a G-code for the 3D printing machine using a second software, e.g., 3D slicer software. Said G-code contains a list of printer-specific commands which allow manufacturing to be carried out. The 3D printing machine converts G-code instructions into hardware operations and adds successive layers of material, thus building the model from a sequence of cross-sections.

Compared with alternative manufacturing tools such as CNC (Computerized Numerical Control) machining, 3D printing provides greater geometric flexibility, greater choice among the materials which can be used in the process, shorter manufacturing times, lower costs, and minimal technical skill requirements on the part of operators. In particular, geometric flexibility is the main reason why 3D printing is best suited for the manufacturing of complex anatomical structures.

However, the use of 3D printing does not allow the manufacturing of models with elastic strength and deformability characteristics corresponding to the different types of organs in the human body. Furthermore, there are economic problems as non-rigid materials that can be 3D printed are expensive. These are also compounded by technical problems in that, to be 3D printed, a material must have hardness values above a minimum value. In other words, with 3D printing, it is not possible to make anatomical models having the structural characteristics of organs, such as liver or kidney.

For example, all methods of 3D printing, including fused filament manufacturing (FFF), VAT photopolymerization, polymer powder sintering, inkjet and polyjet printing (Stratasys) are limited to the use of materials with characteristic hardness typically above 30-50 Shore A. This places limitations on printing different tissues (parenchymal and peritoneal tissues in general, with characteristic hardness between 10 Shore 000 and 80 Shore 00, according to the organ type).

As a result, in many cases, models of organs manufactured by 3D printing are made of materials having a degree of hardness which does not allow emulating the haptic component of organ manipulation when performing surgical procedures, such as palpation, cutting and suturing with such models.

Because of this, although additive manufacturing by means of 3D printing allows the manufacturing of anatomically accurate models of organs, the need is particularly felt to devise a method for manufacturing anatomical models of organs or parts of organs which can reproduce the tactile or haptic response of a patient's organs and tissues so that they can be used in preoperative procedures.

### SUMMARY OF THE INVENTION

Therefore, it is a purpose of the present invention to make available a new method for manufacturing anatomical models adapted to simulate organs or parts of organs of a patient, which allows at least partially overcoming the limitations of the additive printing or 3D printing methods of known types mentioned above.

In particular, the invention is based on the combined use of additive printing, molding methods, material extrusion, and software technologies to obtain patient-specific organ models. Based on the target anatomy to be reproduced, the organ and surrounding structures are parameterized using dedicated software to choose the appropriate tissue-equivalent materials and manufacturing techniques to produce the physical model.

Advantageously, the necessary and sufficient information which can be associated with the organ model and enable its parameterization is contained in a first software library or materials library and a second software library or manufacturing process library. Such first and second software libraries are operationally associated with the program which manages the manufacturing steps of the organ model.

In other words, the aforesaid first and second libraries contain information which can be software employed at the same time to make the anatomical model which simulates the patient's organ in a targeted manner based on the target organ/anatomy and the clinical case to be simulated.

The aforesaid purpose is achieved by means of a method for manufacturing anatomical models adapted to simulate organs or parts of organs of a patient according to claim 1.

Alternative preferred and advantageous embodiments of the aforesaid method are the objects of the dependent claims.

A system for manufacturing anatomical models adapted to simulate organs or parts of organs of a patient according to claim 9 is a further object of the present invention.

It is a further object of the present invention a computer program which implements the method according to claim 11.

### BRIEF DESCRIPTION OF THE DRAWINGS

Further features and advantages of the method for manufacturing anatomical models of the invention will result from the following description of a preferred embodiment given by way of non-limiting example, with reference to the accompanying figures, in which:

FIG. 1 shows a flow chart of a method for-figure shows a manufacturing anatomical models adapted to simulate organs or parts of organs of a patient according to the present invention;

FIG. 2 shows an example of a topographical region in which various tissue components arranged to form layers which create a housing for a given organ, a situation common to a plurality of target anatomical models which can be manufactured by the method of FIG. 1;

FIG. 3a shows a three-dimensional or 3D reconstruction image obtained from scans performed on a patient according to the DICOM (Digital Imaging and Communications in Medicine) standard;

FIG. 3b shows an example of a .stl file which reproduces a kidney obtained from the 3D reconstruction image in FIG. 3a;

FIG. 3c shows an example of a .stl file reproducing a rib cage obtained from the 3D reconstruction image in FIG. 3a;

FIG. 4 diagrammatically shows a system for manufacturing anatomical models adapted to simulate organs or parts of organs of a patient which implements the method of the invention.

Similar or equivalent elements in the aforesaid figures are indicated by means of the same reference numerals.

### DETAILED DESCRIPTION

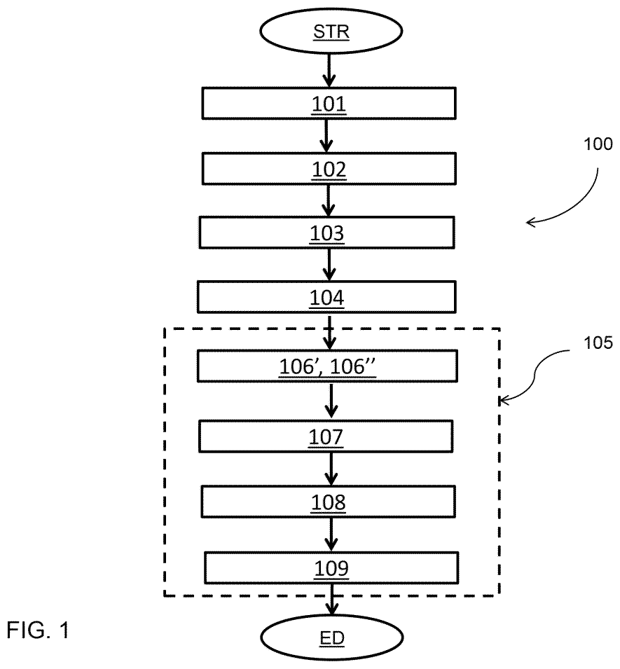

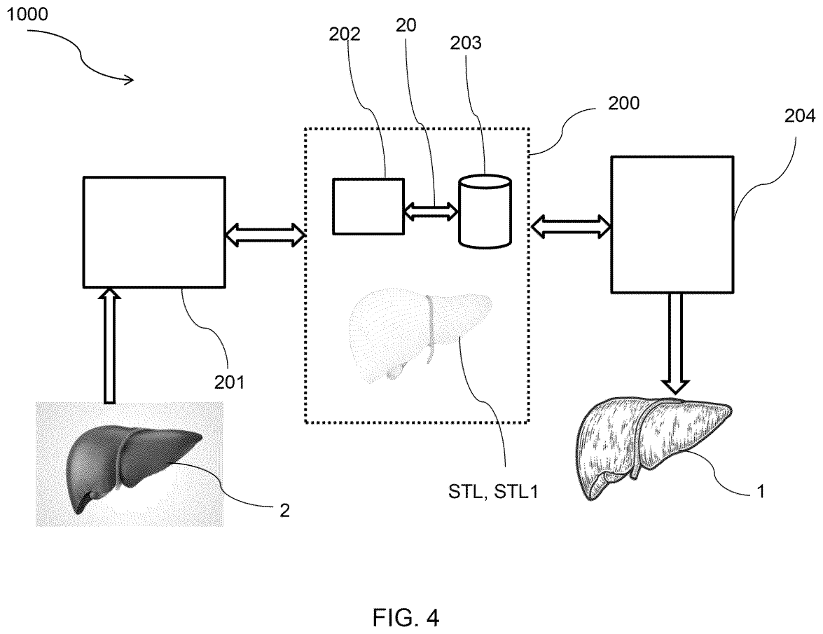

With reference to FIGS. 1, 4, a method for manufacturing anatomical models 1 adapted to simulate organs or parts of organs 2 of a patient according to the present invention is indicated by reference numeral 100. Hereafter said method for manufacturing anatomical models 100 is also simply referred to as the manufacturing method.

In general, the method for manufacturing anatomical models 100 of the invention is performed through a system 1000 comprising hardware/software components, which allow the steps of method 100 to be performed.

In particular, said system 1000 comprises an electronic processing unit 200, i.e., a PC or MAC computer, connected to means 201 of acquiring digital images of organs or parts of organs 2 of a patient. Such means 201 of digital image acquisition are, for example: a CAT (Computerized Axial Tomography) unit, a NMR (Nuclear Magnetic Resonance) unit, radiography/ecography units.

In the example in FIG. 4, the organ 2 of a patient whose anatomical model 1 is being manufactured starting from digital images is the liver.

Such an electronic processing unit 200 comprises at least one processor 202 and one memory block 203 associated with the processor for instruction storage. In particular, said memory block 203 is connected to the processor 202 through a data communication line or bus 20 (e.g., PCI) and consists of a service memory, volatile type (e.g., SDRAM type), and system memory, nonvolatile type (e.g., SSD type).

The software components of the system 1000 preferably comprise but are not limited to computer-aided design (CAD) software, 3D slicer software.

Furthermore, the system 1000 comprises a three-dimensional (3D) printing unit 204 electrically connected to and controlled by the sensor electronic processing unit 200 to manufacture the aforementioned anatomical models 1 based on the processing performed on the digital images.

It is a further object of the present invention a computer program product at any possible level of technical integration detail. Said processor program may include a processor-readable storage medium and including processor-readable program instructions to cause the processor to perform the steps of method 100 of the present invention.

The operational steps of the method 100 for manufacturing anatomical models adapted to simulate organs or parts of organs of a patient through the system 1000 are described in more detail below referring to FIG. 1.

In an embodiment, the electronic processing unit 200 of system 1000 is arranged to execute the codes for an application program implementing the method 100 of the present invention.

In a particular embodiment, the processor 202 is configured to load, in memory block 203, and execute application program codes implementing the method 100 of the present invention.

The manufacturing method 100 comprises symbolic steps of beginning STR and end ED.

The method 100 for manufacturing anatomical models 1 adapted to simulate organs or parts of organs 2 of a patient comprises a step of obtaining information 101 about the anatomical structure of an organ or part of an organ 2 by processing digital images of said organ or part of an organ.

In an embodiment, a step of acquiring representative images of the anatomical structure of an organ or part of an organ 2 is provided by means of the digital image acquisition means 201, which comprise, for example, Computed Axial Tomography, CT scan, Nuclear Magnetic Resonance Imaging, NMR, ultrasound, radiography.

Furthermore, the method 100 comprises a step of selecting or segmenting 102, by means of digital image processing software, of elements of the anatomical structure of an organ or part of an organ 2 from the above-processed images.

Such segmentation software is according to the DICOM (Digital Imaging and Communications in Medicine) standard.

In an embodiment, said step of selecting or segmenting 102 comprises an automatic segmentation and then a manual segmentation having the purpose of refining the model.

Successively, the method 100 allows obtaining 103 a three-dimensional computer model of the anatomical structure of the organ or part of the organ 2 and representing 104 said three-dimensional computer model by means of an STL file having a valid format for use in a three-dimensional (3D) printing process. For example, the chosen format is .stl (STereo Lithography interface format).

Furthermore, the method includes a step of manufacturing 105 of the anatomical model 1 representative of the organ or part of the organ 2 to be simulated by means f the three-dimensional (3D) printing process.

Advantageously, the aforesaid manufacturing step of the method of the invention comprises the following additional steps.

The method 100 involves:
providing 106′ a first software library including digital data representative of a plurality of materials usable for manufacturing the anatomical model 1 of the organ or part of the organ 2 to be simulated;
providing 106″ a second software library including digital data representative of a plurality of methods for manufacturing the anatomical model 1 of the organ or part of the organ 2; said manufacturing methods being usable either alternatively or mutually in combination in the three-dimensional printing process.

As known to a person skilled in the art, a software library is a set of predetermined functions or data structures arranged to be linked to a program through appropriate links.

Furthermore, the method 100 involves a step of selecting 107 at least one material from the first software library and at least one manufacturing method from the second software library based on a representative parameter of a measurement or estimate of the hardness of said organ or part of the organ to be simulated 2, as shown in the third column of Table 1.

It is worth noting that the selection criterion based on which the material library elements and the manufacturing process elements are identified for making haptic models of an organ or anatomical parts is an innovative feature of the method 100 of the present invention.

In particular, the method 100 of the present invention provides that the measurement of the hardness of the organ to be simulated is the discriminating parameter for the selection of materials and manufacturing processes. In other words, the materials and manufacturing processes are jointly chosen from the two software libraries described above-uniquely and automatically-based on said representative parameter of the measurement or estimation of the hardness of the organ (biological consideration) to be reproduced. Therefore, a physical property of the organ to be reproduced is adapted to determine the selection and joint use of the elements of the two software libraries.

The method 100 further includes a step of modifying 108 the STL file representative of the three-dimensional computer model based on said—selected material and manufacturing method to generate an additional STL1 file representative of the modified three-dimensional computer model.

Furthermore, the method 100 includes using 109 the further modified file STL1 to perform the process for manufacturing the anatomical model 1, which simulates the patient's organ or part of the organ 2 by means of a three-dimensional (3D) printing unit 204 of the system 1000.

In particular, a step of converting the additional file STL1 by means of the 3D slicer software to make it interpretable by the printing unit 204 is included.

Based on the present invention, the selection of materials for manufacturing a specific anatomical structure is based on the physical and mechanical characteristics of the organ 2 to be simulated, namely bulk density, hardness, Young's modulus and viscoelastic response (storage and loss moduli).

Other requirements, such as optical transparency, adhesiveness and self-repairing capability, can be taken into consideration when selecting materials.

Based on the morphological details of the target organ 2 to be simulated and the contiguous tissues to be reproduced for a specific clinical application, the selection of materials can be precisely directed toward a subset of suitable candidates representing different structures. For example, while the mechanical properties of the parenchymal component vary among different organs (e.g., brain or visceral organs, such as liver and kidney), peritoneal or fascial structures are characterized by structural uniformity, and the same is true for other organs, e.g., such as muscle and fat.

In an embodiment, the aforesaid materials usable for manufacturing the anatomical model 1 of the organ or part of the organ to be simulated 2 comprise: dielectric gel-based polymer blends, silicones, thermoplastic rubbers, photopolymers.

In another embodiment, the aforesaid methods of manufacturing the anatomical model 1 of the organ or part of the organ to be simulated, comprise: 3D mold forming processes, hot extrusion processes, stereolithographic 3D printing processes.

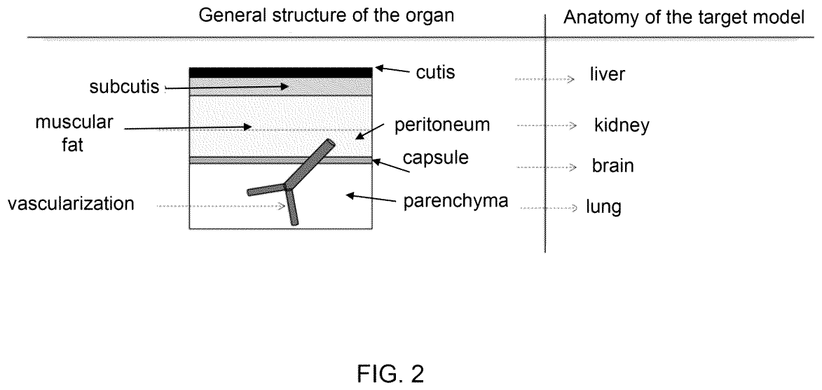

The general selection rules for associating a specific organ 2 and its contiguous anatomical structures with the correct subset of materials and the correct manufacturing techniques (and other combinations thereof) from the first and second software libraries can be diagrammatically illustrated as shown in FIG. 2 and based on Table 1.

TABLE 1

| | | HARDNESS | MANUFACTURING |
|---|---|---|---|
| TISSUE | MATERIALS | (shore) | METHOD |
| Cutis | Silicones and | 10-15 Shore | Molding, coating, |
| | silicone gels, | A | in-tank |
| | hydrogels, | | photopolymerization, |
| | ionogels, organic | | syringe extrusion |
| | and inorganic | | |
| | gels, | | |
| | photopolymers | | |
| Subcutis | Silicones and | 30-40 Shore | Molding, foaming, |
| | silicone gels, | 000 | syringe extrusion, |
| | organic and | | syringe extrusion |
| | inorganic gels, | | assisted by in situ |
| | hydrogel/ionogel | | photopolymerization |
| | and silicone-based | | |
| | polymer foams and | | |
| | polyurethanes, | | |
| | photopolymers | | |
| Adipose and | Silicones and | 30-40 Shore | Molding, syringe |
| muscle tissue | silicone gels, | 000 | extrusion, syringe |
| | hydrogels, | | extrusion assisted |
| | ionogels, organic | | by in situ |
| | and inorganic | | photopolymerization |
| | gels, | | |
| | photopolymers | | |
| Peritoneum | Silicones and | 10-80 Shore | Molding, in-situ |
| | silicone gels, | 00 | photopolymerization, |
| | hydrogels, | | syringe extrusion, |
| | ionogels, organic | | syringe-assisted |
| | and inorganic | | extrusion, in-situ |
| | gels, elastomers | | photopolymerization, |
| | and rubbers, | | in-situ |
| | photopolymers, | | photopolymerization, |
| | thermoplastic | | hot extrusion |
| | rubbers and | | |
| | elastomers | | |
| Capsule | Silicones and | 20 Shore | Coating |
| | silicone gels, | 00-50 Shore | |
| | hydrogels, | A | |
| | ionogels, organic | | |
| | and inorganic | | |
| | gels, | | |
| | photopolymers | | |
| Vasculari- | Silicones and | 20 Shore 00 | Molding, in-tank |
| zation | silicone gels, | -50 Shore | photopolymerization, |
| | hydrogels, | A | syringe extrusion, |
| | ionogels, organic | | syringe-assisted in |
| | and inorganic | | situ |
| | gels, elastomers | | photopolymerization, |
| | and rubbers, | | hot extrusion |
| | photopolymers, | | |
| | thermoplastic | | |
| | rubbers and | | |
| | elastomers | | |
| Parenchyma | Silicones and | 10-80 Shore | Molding, in-tank |
| | silicone gels, | 00 | photopolymerization, |
| | hydrogels, | | syringe extrusion, |
| | ionogels, organic | | syringe-assisted in |
| | and inorganic | | situ |
| | gels, elastomers | | photopolymerization, |
| | and rubbers, | | hot extrusion |
| | photopolymers, | | |
| | thermoplastic | | |
| | rubbers and | | |
| | elastomers | | |
| Pathological | Silicones and | 10 Shore | Molding, syringe |
| structures | silicone gels, | 000-50 | extrusion, in-situ |
| (e.g. tumors, | hydrogels, | Shore A | photopolymerization, |
| injuries, | ionogels, organic | | in-situ |
| etc.) | and inorganic | | photopolymerization- |
| | gels, elastomers | | assisted syringe |
| | and rubbers, | | extrusion, hot |
| | photopolymers, | | extrusion |
| | thermoplastic | | |
| | rubbers and | | |
| | elastomers | | |
| Cartilage | Photopolymers, | 30-100 | In-tank |
| | thermoplastic | Shore A | photopolymerization, |
| | rubbers and | | hot extrusion, |
| | elastomers, | | sintering |
| | thermoplastics, | | |
| | polymer-based | | |
| | sinterable powders | | |
| Bones | Photopolymers, | 10-100 | In-tank |
| | thermoplastics, | Shore D | photopolymerization, |
| | sinterable powders | | hot extrusion, |
| | | | sintering |

In an embodiment, to manufacture anatomical structures comprising:
liver parenchyma, brain, soft tissue of the larynx, skin, subcutaneous muscle, peritoneum, heart, lungs,
the materials selected from the first software library in the step of selecting 107 comprise polymer blends based on dielectric gels and silicones, having a hardness in the range of 35 Shore 000-30 Shore A,
the manufacturing methods selected from the second software library in the step of selecting 107 comprise 3D mold forming processes.

In a further embodiment, to manufacture anatomical structures or also structures with pathological significance comprising:
cartilaginous parts, trachea, thyroid, cricoid, neoformations, such as meningiomas, vascular organs such as arteries and veins,
the materials selected from the first software library in the step of selecting 107 comprise thermoplastic rubbers, having a hardness in the range of 25-35 Shore A,
the manufacturing methods selected from the second software library in the step of selecting 107 comprise hot extrusion processes.

In a further embodiment, to manufacture anatomical structures including: intrahepatic vascular structures, hepatic arteries and veins, and larynx, the materials selected from the first software library in the step of selecting 107 comprise photopolymers, having a hardness between 50 and 80 Shore A,
the manufacturing methods selected from the second software library in the selecting 107 comprise stereolithographic 3D printing processes.

With the present invention, the parameterized anatomical model 1 is physically reproduced using selected appropriately manufacturing technologies including additive printing, molding and extrusion of polymeric materials. The combined use of these approaches, determined by the type of anatomical structure to be manufactured and the software library of materials chosen for manufacturing, is one of the main aspects of constructing a haptic artifact.

With the present invention, direct 3D printing complements other types of molding, the efficiency and speed of the assembly process are linked to the use of a set of standard anatomical parts (anatomical library) always available and usable as the basis for making the final 3D printed model.

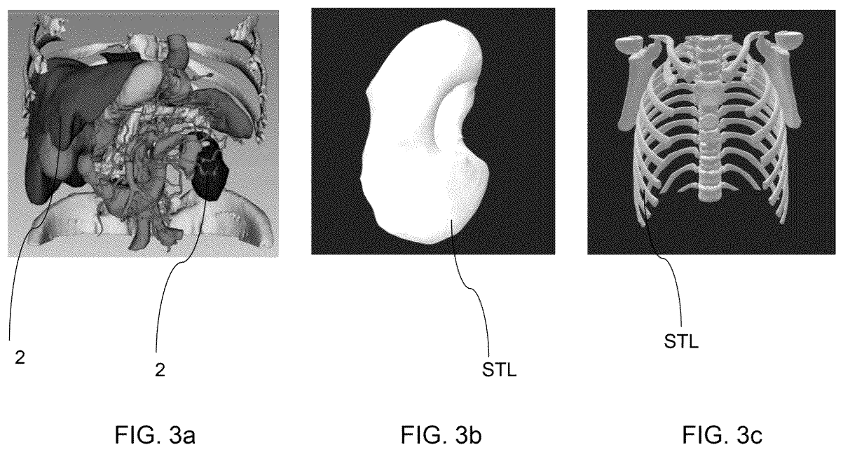

For example, FIG. 3a shows a three-dimensional or 3D reconstruction image obtained from scans performed on a patient according to the DICOM (Digital Imaging and Communications in Medicine) standard. FIGS. 3b and 3c show examples of files in .stl format, depicting a kidney and a rib cage, respectively, obtained from the 3D reconstruction image in FIG. 3a.

In an embodiment, the method 100 comprises the creation of an anatomical software library comprising both the STL file representative of the three-dimensional computer model 1 the additional and STL1 file representative of the modified three-dimensional computer model.

In a further example of embodiment, the method 100 of the invention involves the steps of:
acquiring at least one image of the anatomical model 1, which simulates the patient's organ or part of the organ 2;
comparing at least one acquired image to the images representative of the anatomical structure of said organ or part of the organ of the patient to evaluate the differences between said organ or part of the organ 2 and the anatomical model 1.
   In other words, the image of model 1 can be superimposed on the diagnostic image of the organ 2 after a CT scan of the 3D model itself.

The present invention is based on the combination of additive printing, molding and extrusion of molding polymers for the reproduction of functional characteristics of target organs and anatomical parts. In particular, the following anatomic-functional structures were considered: i) liver (liver parenchyma and vessels); ii) brain (brain tissue, meninges and meningiomas); iii) larynx and trachea; iv) kidneys.

In this context, prototypes were made by virtue of the development of a materials software library and a software library of additive and hybrid manufacturing processes capable of creating complex 3D structures and reproducing the physical characteristics of biological tissues, reproducing features, such as vascularization or spatial anisotropy of the mechanical properties of a given anatomical part.

### EXAMPLE

An example of hybridization of 3D printing and molding techniques to overcome the limitations of known solutions is an anatomical model of a haptic liver, currently being optimized in the Applicant's laboratories. The model is patient-specific and comprises:
a) the parenchyma-produced by molding;
b) the “hollow” vascular system (hepatic artery, portal and vena cava) and bile ducts-produced by VAT photopolymerization;
c) an intrahepatic lesion-produced by molding.

In more detail, the organ is produced by casting the part corresponding to the parenchyma in a dedicated 3D mold, where the vascular and biliary structures, as well as the intrahepatic lesion, are precisely positioned using custom-designed templates.

The parenchyma consists of a platinum-cured silicone-based dielectric gel (hardness 10 Shore 00), the formulation of which was engineered to achieve the tactile response of the organ.

The 3D mold is manufactured from acrylonitrile butadiene styrene using FFF, post-processed with acetone smoothing.

Before the gel is poured and cured in the mold, the vascularization, biliary tree, and intrahepatic lesion are placed in the mold and physically positioned by means of appropriately made templates and reference points. UV stereolithography at 405 nm was used to manufacture the vascular and biliary structures, while the lesion was printed in a model manufactured using LCD-UV 3D printing and consists of a hard 20 Shore 00 elastomer. The manufactured model results in an anatomically realistic replica of the patient's organ, with appropriate tactile (haptic) feedback, as evaluated by several experts in liver and vascular surgery.

The method 100 for manufacturing anatomical models 1 adapted to simulate organs or parts of organs 2 of a patient of the present invention and the related system 1000 have numerous advantages and achieve their intended purposes.

The suggested method overcomes the challenge of modeling tactile response by manufacturing a 3D model of an anatomical structure based on one or more additive methods configured to combine different materials in the 3D model.

In an example, the 3D model can be manufactured by a 3D printer with a base material and one or more “soft” materials used for molding.

This approach allows different materials to be combined in a 3D model of an organ, thus enabling a health care provider to detect a realistic tactile or haptic response.

The solution suggested by the present invention, which combines the selection of one or more different materials from a first library and one or more manufacturing methods from a second library, allows for faster, more efficient and economically sustainable manufacturing of artifacts than other existing manufacturing methods based on or referring only to the use of material libraries.

Furthermore, by enabling an overlay of the original diagnostic image of the organ 2 with the image obtained from three-dimensional model 1, differences can be quantitatively evaluated in a closed-loop process that improves the manufacturing method.

Furthermore, by making an overlay of images of the original model and the three-dimensional model 1, e.g., a diagnostic image of the organ 2 superimposed on an image of the 3D model, a physician can evaluate the differences.

For the sake of simplicity, the aspects of the present invention are discussed with respect to diagnostic images and 3D models of a human “organ” 2, however, this should not be considered a limitation.

In this regard, aspects of the present invention are also applicable to typical anatomical features of organs of mammals and to anatomical features of parts other than organs such as, e.g., bones, tendons, ligaments, muscles and other anatomical structures.

Furthermore, the aspects of the present invention are applicable to combinations of any of the aforesaid anatomical features, such as, e.g., a central section of the patient including organs (e.g., colon, liver, lungs), bones (e.g., ribs, spine), muscles (e.g., abdominal muscles), and/or other anatomical features.

Finally, although explicit reference is made to single images (e.g., a diagnostic image, a model image) it is worth emphasizing that the method of the invention is applicable to numerous images. For example, a CT image can illustrate an anatomical feature in two dimensions for a specific cross-sectional location, while a plurality of CT scans can be used to collectively illustrate the anatomical feature in three dimensions.

To meet contingent needs, those skilled in the art may make changes and adaptations to the embodiments of the method described above or can replace elements with others which are functionally equivalent, without departing from the scope of the following claims. All the features described above as belonging to a possible embodiment can be implemented independently of the other embodiments described.

What is claimed is:

1. A method for manufacturing anatomical models adapted to simulate organs or parts of organs of a patient, comprising the steps of:
obtaining information on the anatomical structure of an organ or part of organ by processing digital images of said organ or part of organ;
selecting, by digital image processing software, elements of the anatomical structure of said organ or part of organ from the processed images to obtain a three-dimensional computerized model of the anatomical structure of said organ or part of organ;
representing said three-dimensional computerized model by a file having a valid format for use in a three-dimensional, 3D printing process;
manufacturing the anatomical model representative of said organ or part of organ to be simulated by the three-dimensional printing process, said manufacturing step comprises the further steps of:
providing a first software library including digital data representative of a plurality of materials usable for manufacturing the anatomical model of the organ or part of organ to be simulated;
providing a second software library including digital data representative of a plurality of methods for manufacturing the anatomical model of the organ or part of organ to be simulated, said manufacturing methods being usable either alternatively or mutually in combination in the three-dimensional printing process;
selecting at least one material from said first software library and at least one manufacturing method from said second software library based on a parameter representative of a hardness measurement of said organ or part of organ to be simulated;
modifying the file representative of the three-dimensional computerized model based on said at least one material and said at least one manufacturing method selected to generate a further file representative of the modified three-dimensional computerized model;
using the further modified file to perform the process for manufacturing the anatomical model which simulates the patient's organ or part of organ by a three-dimensional 3D, printing unit.

2. A method for manufacturing anatomical models according to claim 1, wherein said materials usable for manufacturing the anatomical model of the organ or part of organ to be simulated comprise: dielectric gel-based polymer blends, silicones, thermoplastic rubbers, photopolymers.

3. The method for manufacturing anatomical models according to claim 1, wherein said methods of manufacturing the anatomical model of the organ or part of organ to be simulated, comprise: 3D mold forming processes, hot extrusion processes, stereolithographic 3D printing processes.

4. The method for manufacturing anatomical models according to claim 1, wherein in order to manufacture anatomical structures comprising:
liver parenchyma, brain, soft tissue of the larynx, skin, subcutaneous muscle, peritoneum, heart, lungs,
the materials selected from the first software library in the selecting step comprise polymer blends based on dielectric gels and silicones, having a hardness in the range of 35 Shore000ShoreA,
the manufacturing methods selected from the second software library in the selecting step comprise 3D mold forming processes.

5. The method for manufacturing anatomical models according to claim 1, wherein in order to manufacture anatomical structures comprising:
cartilage parts, trachea, thyroid, cricoid, vascular organs such as arteries and veins, neoformations such as meningiomas,
the materials selected from the first software library in the selecting step comprise thermoplastic rubbers, having hardness in the range of 25-35 ShoreA,
the manufacturing methods selected from the second software library in the selecting step comprise hot extrusion processes.

6. The method for manufacturing anatomical models according to claim 1, wherein in order to manufacture anatomical structures comprising:
intrahepatic vascular structures, arteries and veins, larynx,
the materials selected from the first software library in the selecting step comprise photopolymers, having hardness between 50 and 80 Shore A,
the manufacturing methods selected from the second software library in the selecting step comprise stereolithographic 3D printing processes.

7. The method for manufacturing anatomical models according to claim 1, further comprising a step of generating an anatomical software library comprising said file representative of the three-dimensional computerized model and said further file representative of the modified three-dimensional computerized model.

8. The method for manufacturing anatomical models according to claim 1, further comprising the steps of:
acquiring at least one digital image of the anatomical model which simulates the patient's organ or part of organ;
comparing said at least one acquired image to the digital images representative of the anatomical structure of said organ or part of organ to evaluate the differences between said organ or part of organ and said anatomical model.

9. A system for manufacturing anatomical models adapted to simulate organs or parts of organs of a patient, comprising:
acquisition means for acquiring digital images of organs or parts of organs of a patient;
an electronic processing unit connected to said digital image acquisition means for receiving and processing said digital images;
a three-dimensional 3D, printing unit controlled by the electronic processing unit to manufacture the aforementioned anatomical models based on the processing of digital images,
wherein said electronic processing unit comprises at least one processor and a memory block associated with the processor for storing instructions, said processor and said memory block being configured to carry out the steps of the method according to claim 1.

10. The system for manufacturing anatomical models according to claim 9, wherein said acquisition means for acquiring digital images are selected from the group consisting of: a (computerized Axial Tomography) unit, CAT, a (Nuclear Magnetic Resonance) unit, NMR, a radiography unit, an ultrasound unit.

11. A computer program comprising an application code executable by an electronic processing unit to implement the method according to claim 1.
